(19) United States
(12) Patent Application Publication
(10) Pub. No.: US 2023/0341365 A1
(43) Pub. Date: Oct. 26, 2023

(54) METHODS AND KITS FOR DETECTING PEROXYMONOSULFATES

(71) Applicant: CYPRUS UNIVERSITY OF TECHNOLOGY, Limassol (CY)

(72) Inventors: Maria ANTONIOU, Limassol (CY); Eleni KELIRI, Limassol (CY); Nikoletta TSIARTA, Limassol (CY)

(21) Appl. No.: 18/026,738

(22) PCT Filed: Sep. 16, 2021
(86) PCT No.: PCT/EP2021/075500

§ 371 Date: Mar. 16, 2023

(30) Foreign Application Priority Data
Sep. 17, 2020 (CY) 202000002

(51) Int. Cl. G01N 31/22; G01N 33/18; G01N 21/78

(52) U.S. Cl. CPC G01N 31/228; G01N 33/182; G01N 21/78

(57) ABSTRACT

Provided herein are methods and kits for detecting or quantifying a peroxymonosulfate (HS0₅₋) in an aqueous solution via electromagnetic radiation absorbance measurement.

### BACKGROUND

Detection and quantification of water solutes is useful to understand the chemical profile of water samples, especially when the source water is for general human use where health protocols or maximum allowable limits may apply. With this in mind, it is helpful to take precautionary measurements to safeguard health from microbial or chemical sources of contamination. Chlorine-based and chlorine-free agents for disinfecting water are available. Chlorine-free disinfection agents, i.e. non-bleaching based oxidants, may be preferable to chlorine-based disinfection agents because chlorine-based disinfection agent by-products can be toxic. Non-bleaching based oxidants may be preferred for shock oxidation and disinfection of pools and spas. Non-bleaching oxidizers, such as peroxymonosulfate (PMS; i.e. K⁺HSO₅⁻), can be used for disinfection, for example, when foreign substances or microorganisms such as urine, bacteria, algae, viruses, fungus, molds, sun-blocking agents, such as sun-tan oils or creams, or sweat are present in water.

Determining the oxidant demand for a PMS in a specific water source, which may be in need of disinfection, is difficult without the ability to monitor the water through detection and quantification of the water's composition, specifically, the presence of a PMS. This may lead to overdosing or underdosing with a disinfection agent in order to comply with health or safety protocols.

Thus, there is a need to detect or quantify the presence of solutes, i.e. added oxidants such as a PMS, in water in real time, such as within seconds or minutes. The ability to do so allows for mitigation of accumulating additional water solutes and limits the hazards of human exposure to high concentrations of disinfection agents, including PMSs.

### SUMMARY

Provided herein are methods of detecting or quantifying HSO₅⁻ in an aqueous solution. In some embodiments, the methods comprise comparing an electromagnetic radiation absorbance measurement of a mixture of a reagent solution and a sample to a standard calibration profile to determine the presence or amount of HSO₅⁻ in the sample. The absorbance measurement includes at least a portion of the wavelength range from about 380-450 nm. In other embodiments, the reagent solution includes para-nitrophenyl boronic acid and an aqueous vehicle. In other embodiments, the reagent solution includes para-nitrophenyl boronic acid, a buffer, and an aqueous vehicle.

In another aspect, provided herein are kits for detecting or quantifying HSO₅⁻ in an aqueous solution, comprising: a reagent solution, which includes para-nitrophenyl boronic acid and an aqueous vehicle; and instructions for use.

### BRIEF DESCRIPTION OF THE DRAWINGS

FIG. 1 shows a diagram of a general method for quantifying or determining the presence of HSO₅⁻ in an aqueous sample as described in Example 1.

FIG. 2 shows the quantification of HSO₅⁻ in lake water containing two different concentrations of HSO₅⁻ as described in Example 4 (top line: 5 mg/L; bottom line: 2 mg/L).

FIG. 3 shows the quantification of HSO₅⁻ in Type I water (i.e. Milli-Q or extra pure water) containing two different concentrations of HSO₅⁻ as described in Example 4 (top line: 5 mg/L; bottom line: 2 mg/L).

FIG. 4 shows the quantification of HSO₅⁻ in river water containing three different concentrations of HSO₅⁻ as described in Example 4 (top line: 5 mg/L; upper middle line: 3 mg/L; lower middle line: 1 mg/L; bottom line: control).

### DETAILED DESCRIPTION

### Definitions

Listed below are definitions of various terms used to describe the methods and kits provided herein. These definitions apply to the terms as they are used throughout this specification and the claims, unless otherwise limited in specific instances, either individually or as part of a larger group.

Unless defined otherwise, all technical and scientific terms used herein generally have the same meaning as commonly understood by one of ordinary skill in the art to which the methods and kits provided herein pertain. Generally, the nomenclature used herein and the laboratory procedures in chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed compositions and methods.

As used herein, the term “peroxymonosulfate” or its acronym “PMS” refers to a compound that includes the anion or moiety HSO₅⁻. Thus, peroxymonosulfates include, but are not limited to alkali salts of HSO₅⁻. Peroxymonosulfates also include HSO₅⁻. Peroxymonosulfates also include, more generally, metal salts of HSO₅⁻, which may be of the formula Mⁿ⁺(HSO₅⁻)ₚ, where M is a metal element, n is 1, 2, or 3, or corresponds to the charge state of the metal element, and p is 1, 2, 3, 4, 5, 6, or 7. n and p may be the same, or n and p may be different. Peroxymonosulfates also include organic cations and inorganic cations. For example, quaternary ammonium cations or amino-type cations, among other organic cations, are contemplated.

### Methods

Provided herein are methods of detecting or quantifying HSO₅⁻ in aqueous solutions. Also provided are kits comprising reagents useful for detecting or quantifying HSO₅⁻ in aqueous solutions.

Peroxymonosulfates react with para-nitrophenylboronic acid (p-NPBA) to form para-nitrophenol. Para-nitrophenol has a pKₐ of 7.15 in water. When deprotonated, para-nitrophenol has a high absorbance at 405 nm resulting in a solution of deprotonated para-nitrophenol being yellow in appearance.

In some embodiments, provided herein are methods of detecting or quantifying a peroxymonosulfate (i.e. HSO₅⁻) in an aqueous solution. The aqueous solution may be a water sample from any source, including a laboratory water source, a municipal water source, a surface water source, a ground-water source, or an aquifer source. In some embodiments, the surface water source is an ocean, a lake, a river, an estuary, a delta, a stream, a fountain, or a pool. In some embodiments, the pool may be a swimming pool, a soaking pool, a tub, or a spa.

In some embodiments, provided herein are methods of detecting or quantifying HSO₅⁻ in an aqueous solution, comprising:
comparing an electromagnetic radiation absorbance measurement of a mixture of a reagent solution and a sample to a standard calibration profile to determine the presence or amount of HSO₅⁻ in the sample;
wherein the absorbance measurement includes at least a portion of the wavelength range from about 380-450 nm; and
the reagent solution includes para-nitrophenyl boronic acid, a buffer, and an aqueous vehicle.

In some embodiments, the methods provided herein are performed under dark conditions, such as in the substantial or nearly complete absence of visible light.

The methods provided herein may be performed with or without a buffer. As a PMS converts para-nitrophenylboronic acid to para-nitrophenol, the accumulation of para-nitrophenol serves as both an indicator, by its yellow color, of the presence of the PMS and as a buffer since para-nitrophenol has a pKₐ of 7.15 in water. Thus, in some embodiments, the reagent solution of the methods provided herein does not include a buffer.

Thus, in some embodiments, provided herein are methods of detecting or quantifying HSO₅⁻ in an aqueous solution, comprising:
comparing an electromagnetic radiation absorbance measurement of a mixture of a reagent solution and a sample to a standard calibration profile to determine the presence or amount of HSO₅⁻ in the sample;
wherein the absorbance measurement includes at least a portion of the wavelength range from about 380-450 nm; and
the reagent solution includes para-nitrophenyl boronic acid, and an aqueous vehicle.

In some embodiments of these methods, the buffer has a pKₐ of at least 7.0. In some embodiments, the buffer has a pKₐ of at least 7.5. In some embodiments, the pKₐ of the buffer is with respect to a temperature of about 0° C. to about 120° C. In some embodiments, the pKₐ of the buffer is the buffer's pKₐ at about 20-25° C.

In some embodiments, the buffer includes a buffering compound selected from a phosphate, a borate, a pyrophosphate, piperazine, glycine, a bicarbonate, methylamine, or piperidine. In some embodiments, the buffer includes a buffering compound selected from Table 1.

In some embodiments, the buffer includes more than one, i.e. two or three, buffering compounds described herein. The pKₐ of a buffer system including more than one buffering compounds is a combination of the individual pKₐ values of each buffering compound in the mixed buffering system. Thus, in some embodiments, the pKₐ of the buffer is at or above the pKₐ of para-nitrophenol even though the buffer may include a first buffering compound having an individual pKₐ at or below the pKₐ of para-nitrophenol and a second buffering compound having an individual pKₐ at or above the pKₐ of para-nitrophenol.

In some embodiments, the buffer is present in the reagent solution in a concentration of about 5.0 mM to about 200 mM. In some embodiments, the buffer is present in the reagent solution in a concentration of about 5.0 mM to about 100 mM. In some embodiments, the buffer is present in the mixture in a concentration of about 2.0 mM to about 50 mM. In some embodiments, the buffer is present in the mixture in a concentration of about 2.0 mM to about 25 mM. In some embodiments, the buffer is present in the mixture in a concentration of about 1.0 mM to about 10 mM.

TABLE 1
Buffers for use as described herein.

| Buffering compound | pKₐ at 20° C. | pKₐ at 25° C. |
|---|---|---|
| Phosphate, monobasic | 7.21 | 7.20 |
| HEPES | 7.55 | 7.48 |
| Triethanolamine | 7.66 | 7.76 |
| Tricine | 8.15 | 8.05 |
| Tris | 8.30 | |
| Bicine | 8.35 | 8.26 |
| Borate | 9.24 | 9.23 |
| Pyrophosphate, tetrabasic | | 9.32 |
| Piperazine | | 9.73 |
| Glycine | | 9.78 |
| Bicarbonate | 10.3 | 10.33 |
| Methylamine | | 10.66 |
| Piperidine | | 11.12 |
| Phosphate, dibasic | 12.32 | 12.33 |

In some embodiments, the reagent solution has a pH of not less than about 7.5. In some embodiments, the reagent solution has a pH of about ±0.5 pH of the pKₐ of the buffer. In some embodiments, the pH is at least 7.15. In some embodiments, the pH is at least 8.5. In some embodiments, the pH is about 9.2, 10.3, 11.1, or 12.4. In some embodiments, the pH is at least 9.5.

In some embodiments, the para-nitrophenyl boronic acid is present in the reagent solution in a concentration of from 0.1 mM to less than 5.0 mM. In some embodiments, the para-nitrophenyl boronic acid is present in the reagent solution in a concentration of from 0.5 mM to less than 5.0 mM. In some embodiments, the para-nitrophenyl boronic acid is present in the reagent solution in a concentration of from 1.0 mM to less than 4.0 mM. In some embodiments, the para-nitrophenyl boronic acid is present in the reagent solution in a concentration of about 2.0 mM.

In some embodiments, the reagent solution and the sample are mixed in a container that substantially blocks transmission of electromagnetic radiation having a wavelength of about 10-750 nm. In some embodiments, the reagent solution and the sample are mixed in a container that substantially blocks transmission of electromagnetic radiation having a wavelength of about 200-500 nm. In some embodiments, the reagent solution and the sample are mixed in a container that substantially blocks transmission of electromagnetic radiation having a wavelength of about 350-500 nm. In some embodiments, the container is a container surrounding the vessel in which the sample is mixed. In some embodiments the mixing vessel and the container are the same object. In some embodiments, the container and the mixing vessel are not the same object. In some embodiments, the container is made of cardboard, metal, plastic, or paper. In some embodiments, the container is made of a glass. In some embodiments, the container is made of an amber colored glass. In some embodiments, it may be effective where the container is organic matter, such as a leaf, a grass, or dirt, that, when wrapped around or surrounding the mixing vessel, protects the mixing vessel from visible or ultraviolet light.

In some embodiments, the reagent solution and the sample are mixed in a dark environment. In some embodiments, the reagent solution and the sample are mixed in a dark room or a dark chamber.

In some embodiments, the container or the mixing vessel is a plastic container, a glass container, a tinted glass container, a metal container, or a paper container.

In some embodiments, the method is capable of detecting HSO₅⁻ in the sample that has a concentration of about 100 μg/L or more of HSO₅⁻. In some embodiments, the sample has a concentration of about 0.1 ppm or more of HSO₅⁻. In some embodiments, the method is capable of quantifying HSO₅⁻ in the sample that has a concentration of about 500 μg/L or more of HSO₅⁻.

In some embodiments, the absorbance measurement is performed visually. In some embodiments, the absorbance measurement is obtained and compared with a color-coded reference chart or wheel. In some embodiments, the absorbance measurement is performed mechanically.

In some embodiments, the absorbance measurement includes a wavelength of about 405 nm.

In some embodiments, the comparison is performed within 60 seconds of mixing the reagent solution and the sample. In some embodiments, the comparison is performed within 10 minutes of mixing the reagent solution and the sample. In some embodiments, the comparison is performed within 30 minutes of mixing the reagent solution and the sample. In some embodiments, the comparison is performed within 60 minutes of mixing the reagent solution and the sample. In some embodiments, the comparison is performed within 4 hours of mixing the reagent solution and the sample. In some embodiments, the comparison is performed within 8 hours of mixing the reagent solution and the sample. In some embodiments, the comparison is performed within 12 hours of mixing the reagent solution and the sample. In some embodiments, the comparison is performed within 24 hours of mixing the reagent solution and the sample.

In some embodiments, the buffer does not include carbonate or bicarbonate.

### Kits

In some embodiments, provided herein are kits for detecting or quantifying a peroxymonosulfate (i.e. HSO₅⁻) in an aqueous solution, comprising:
a reagent solution, which includes para-nitrophenyl boronic acid, a buffer, and an aqueous vehicle; and
instructions for use.

In some embodiments, the reagent solution is in a container that partially or completely blocks transmission of visible light, ultraviolet light, or visible and ultraviolet light, and the kit further includes a color-coded reference corresponding to HSO₅⁻ concentrations.

### EXAMPLES

The following Examples further illustrate aspects of the compositions and methods provided herein. However, these Examples are in no way a limitation of the teachings or disclosure as set forth herein. These Examples are provided for illustration purposes.

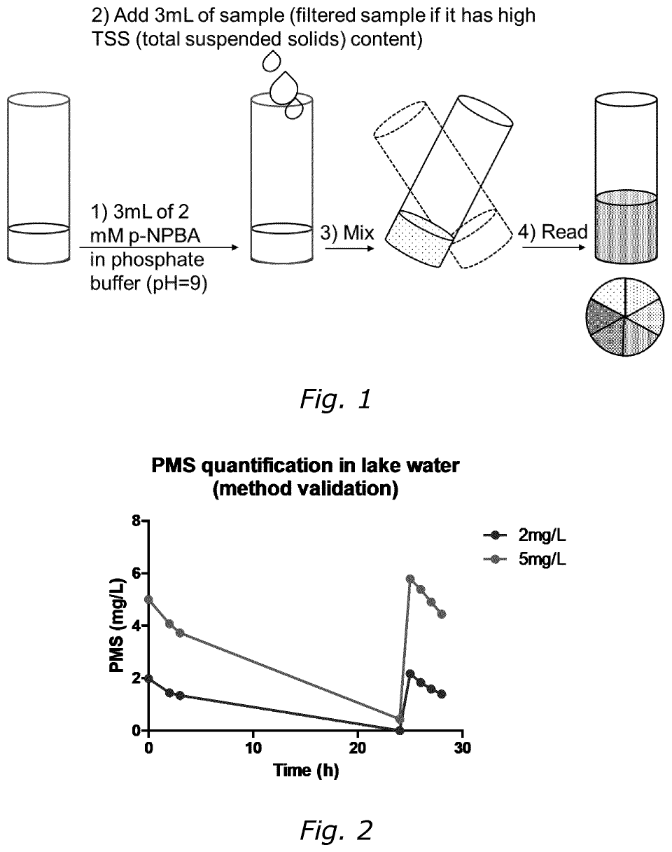

### Example 1: General Procedure Shown in FIG. 1

A water sample was filtered or centrifuged, or both, and decanted to remove excessive total suspended solids. 3 mL of 2 mM para-nitrophenylboronic acid (p-NPBA) in 5 mM phosphate buffer at pH 9.0 is mixed with 3 mL of the filtered water sample, agitated, and the absorbance at 405 nm was observed. A yellow colored solution indicates that the water sample included HSO₅⁻ since p-NPBA and HSO₅⁻ gives different shades of yellow having a maximum absorbance at 405 nm. The reagent and reaction solution are photosensitive, and should be kept away from light for a more accurate reading. Exposing the reaction solution to sunlight for 10 minutes will result in a saturated yellow solution that does not depend on the presence of any potential HSO₅⁻. The solution's color is compared with a color-coded reference or the absorbance of the solution is read and compared to a standard calibration profile.

### Example 2: Stability of Reaction Kinetics

In a quartz cuvette, under dark conditions, 2 mL of aqueous 50 μM HSO₅⁻ sample was mixed with 2 mL of 2 mM p-NPBA in water. The cuvette was left inside the spectrophotometer, and absorbance measurements were obtained every hour up to 8 hours, and at 24, 26, and 28 hours. Table 2 shows the absorbance readings, which indicate the reaction's absorbance profile is stable over multiple hours (i.e. for at least one day).

TABLE 2
Reaction Kinetics

| Time (hours) | Average Absorbance at 405 nm |
|---|---|
| 0.0 | 0.44 |
| 1.0 | 0.45 |
| 2.0 | 0.45 |
| 3.0 | 0.45 |
| 4.0 | 0.45 |
| 5.0 | 0.45 |
| 6.0 | 0.45 |
| 7.0 | 0.45 |
| 8.0 | 0.45 |
| 24.0 | 0.46 |
| 26.0 | 0.46 |
| 28.0 | 0.45 |

### Example 3: Calibration Curve

Standard samples of HSO₅⁻ were reacted with p-NPBA to generate a calibration curve and a standard line was fit to the data. Results are shown in Table 3. The minimum detection limit (MDL) was determined to be 0.115 mg/L (standard deviation (0.0116 mg/L)*9.925), and the limit of quantification (LOQ) was determined to be 0.575 mg/L (5*MDL). If a test sample initially results in an absorbance at 405 nm that is higher than the LOQ, then the source sample may be diluted with water and tested. The dilution factor would be taken into account when determining the actual concentration of HSO₅⁻ in the source sample.

TABLE 3
Calibration

| HSO₅⁻ (mg/L) | Average Absorbance at 405 nm |
|---|---|
| 0.0 | 0.100 |
| 0.1 | 0.120 |
| 0.2 | 0.142 |
| 0.8 | 0.268 |
| 2.0 | 0.558 |
| 5.0 | 1.196 |
| 10.0 | 2.248 |
| Slope | 0.2154 ± 0.001554 |
| Y-intercept when X = 0.0 | 0.1048 ± 0.006689 |

### Example 4: Method Validation

Eight samples were used to validate the method. The first and second samples were lake water that included 2 mg/L or 5 mg/L HSO₅⁻, respectively. The third and fourth sample were Type I water (i.e. Milli-Q or extra-pure water) that was supplemented with 2 mg/L or 5 mg/L HSO₅⁻, respectively. The fifth, sixth, seventh, and eight samples were river water, and river water that included 1 mg/L HSO₅⁻, 3 mg/L HSO₅⁻, or 5 mg/L HSO₅⁻, respectively.

The water samples were collected and spiked with HSO₅⁻, then allowed to sit exposed to light and air until a portion of the sample was removed for analysis. After 24 hours, samples 1-4 were spiked with a second amount of HSO₅⁻. For example, lake water sample 1 was spiked to a final concentration of 2 mg/L HSO₅⁻ at time zero, and spiked to a final concentration of 2 mg/L HSO₅⁻ at 24 hours.

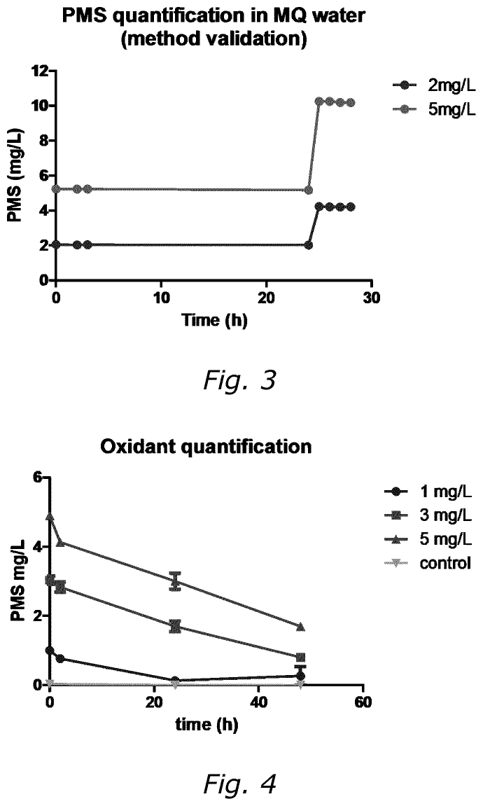

HSO₅⁻ is consumed by organic matter in source waters, and therefore a decrease in the amount of HSO₅⁻ in a spiked water sample that contains organic matter is expected. This effect was observed in lake water samples 1 and 2, as shown in FIG. 2 (0-24 hours), which corresponds to the first and second samples. A lack of organic matter in a water sample permits the HSO₅⁻ to persist without degradation or consumption, even after 24 hours of exposure to light an air. This effect was observed in Type I water samples 3 and 4, as shown in FIG. 3 (0-24 hours). The consumption of HSO₅⁻ by organic matter in river water was observed in river water samples 6-8, as shown in FIG. 4 (0-48 hours).

It is to be understood that wherever values and ranges are provided herein, all values and ranges encompassed by these values and ranges, or a combination of these values and ranges, are meant to be encompassed within the scope of the aspects and embodiments provided herein. Moreover, all values that fall within these ranges, as well as the upper or lower limits of a range of values, are also contemplated by the present application.

Those skilled in the art will recognize, or be able to ascertain using no more than routine experimentation, numerous equivalents to the specific procedures, embodiments, claims, and examples described herein. Such equivalents are intended to be encompassed by the following claims.

1. A method of detecting or quantifying HSO₅⁻ in an aqueous solution, comprising:
comparing an electromagnetic radiation absorbance measurement of a mixture of a reagent solution and a sample to a standard calibration profile to determine the presence or amount of HSO₅⁻ in the sample;
wherein the absorbance measurement includes at least a portion of the wavelength range from about 380-450 nm; and
the reagent solution includes para-nitrophenyl boronic acid, a buffer, and an aqueous vehicle.

2. The method of claim 1, wherein the reagent solution has a pH of not less than about 7.5.

3. The method of claim 1, wherein the reagent solution has a pH of about ±0.5 pH of the pKₐ of the buffer.

4. The method of claim 3, wherein the pH is at least 7.15.

5. The method of claim 3, wherein the buffer has a pKₐ of at least 7.5.

6. The method of claim 2, wherein the pH is at least 8.5 or at least 9.5.

7. The method of claim 2, wherein the pH is about 9.2, 10.3, 11.1, or 12.4.

8. (canceled)

9. The method of claim 1, wherein the buffer includes a phosphate, a borate, a pyrophosphate, piperazine, glycine, a bicarbonate, methylamine, or piperidine.

10. The method of claim 1, wherein the para-nitrophenyl boronic acid is present in the reagent solution in a concentration of from 0.5 mM to less than 5.0 mM.

11. The method of claim 1, wherein the para-nitrophenyl boronic acid is present in the reagent solution in a concentration of from 1.0 mM to less than 4.0 mM.

12. The method of claim 1, wherein the para-nitrophenyl boronic acid is present in the reagent solution in a concentration of about 2.0 mM.

13. The method of claim 1, wherein the buffer is present in the reagent solution in a concentration of about 5.0 mM to about 200 mM.

14. The method of claim 1, wherein the buffer is present in the reagent solution in a concentration of about 5.0 mM to about 100 mM.

15. The method of claim 1, wherein the buffer is present in the mixture in a concentration of about 2.0 mM to about 50 mM.

16. The method of claim 1, wherein the reagent solution and the sample are mixed in a container that substantially blocks transmission of electromagnetic radiation having a wavelength of about one of:
10-750 nm;
200-500 nm; or
350-500 nm.

17-18. (canceled)

19. The method of claim 1, wherein the method is capable of detecting HSO₅⁻ in the sample that has a concentration of about 100 μg/L or more of HSO₅⁻.

20. The method of claim 1, wherein the sample has a concentration of about 0.1 ppm or more of HSO₅⁻.

21. The method of claim 1, wherein the method is capable of quantifying HSO₅⁻ in the sample that has a concentration of about 500 μg/L or more of HSO₅⁻.

22. The method of claim 1, wherein the absorbance measurement is performed visually or mechanically.

23. (canceled)

24. The method of claim 1, wherein the absorbance measurement includes a wavelength of about 405 nm.

25. The method of claim 1, wherein the comparison is performed within 60 seconds of mixing the reagent solution and the sample.

26. The method of claim 1, wherein the buffer does not include carbonate or bicarbonate.

27. A kit for detecting or quantifying HSO₅⁻ in an aqueous solution, comprising:
a reagent solution, which includes para-nitrophenyl boronic acid, a buffer, and an aqueous vehicle; and
instructions for use.

28. The kit of claim 27, wherein the reagent solution is in a container that partially or completely blocks transmission of visible light, ultraviolet light, or visible and ultraviolet light, and the kit further includes a color-coded reference corresponding to HSO₅⁻ concentrations.
